(19) United States
(12) Patent Application Publication
(10) Pub. No.: US 2009/0253126 A1
(43) Pub. Date: Oct. 8, 2009

(54) Biomarker Detection Assays

(75) Inventors: Paul Ciaccio, Wilmington, DE (US); Joseph Milano, Wilmington, DE (US); Francois Pognan, Cheshire (GB)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(73) Assignee: ASTRAZENECA AB, Sodertalje (SE)

(21) Appl. No.: 10/598,339

(22) PCT Filed: Feb. 21, 2005
(86) PCT No.: PCT/SE2005/000234

§ 371 Date: Aug. 24, 2006

Related U.S. Application Data
(60) Provisional application No. 60/547,734, filed on Feb. 25, 2004.

(51) Int. Cl. C12Q 1/68; C12Q 1/02

(52) U.S. Cl. 435/6; 435/29

(57) ABSTRACT

A method for identifying compounds capable of modulating the Notch pathway. The test compound is either administered to an animal or is brought into contact with a cell in culture, after which the expression of the basic helix-loop-helix transcription factor Math1 in a sample from said animal or cell is directed. A change in Math1 expression in the presence of said test compound as compared to in the absence of said compound indicates that said compound modulates the Notch pathway.

### CROSS-REFERENCE TO RELATED APPLICATIONS

This application is related to U.S. Provisional Application 60/547,734, filed Feb. 25, 2004, which is incorporated herein by reference in its entirety.

### FIELD OF THE INVENTION

The present invention relates to assays for drug-induced modulation of the Notch pathway by the detection of expression of Math1, the mammalian homologue of Drosophila atonal (ATH1), a basic helix-loop-helix (bHLH) transcription factor.

### BACKGROUND

The identification of biomarkers of toxicity validated in animal models can mitigate risk of clinical toxicity. Toxicity biomarker screens permit (1) the evaluation of safety parameters that are expected by both regulators and clinicians, and (2) the careful design of preclinical and clinical studies, including proof of species difference studies; careful dose escalation studies; the safe progression from preclinical studies to first-in-man studies where therapeutic margins of animal no effect levels (NOELS) and anticipated human efficacy doses are narrow; and early withdrawal of a drug candidate if clinical toxicity is approached. Additionally, toxicity biomarkers can be employed in counter-screening of drug project back-ups.

Notch1 is an integral membrane protein that governs a wide array of cell differentiation pathways. The activation or suppression of a given cell fate is orchestrated by the balance between expression of Notch and the expression of the Notch ligand, Delta1 (Lewis, 1998, Cell Dev. Biol., 9:583-589). Perturbation of the Notch1 pathway has been shown to have deleterious effects (Haddon et al., 1998, Development 125:4637-4644). Knockout (KO) studies of elements downstream of the Notch signal, such as the transcriptional regulators Hes1 and Math1, illustrate this pathway's role in the regulation of stem cell differentiation (Jensen et al., 2000, Nat. Gen. 24:36-44; Yang et al., 2001, Science 294:2155-2158).

The Notch signal is mediated by a terminal intramembranous cleavage by γ-secretase releasing the Notch intracellular domain (NICD). The NICD is shuttled to the nucleus where it recruits several co-factors that initiate gene transcription of several elements including Hes1 (Baron, 2003, Cell Dev. Biol., 14:113-119). Hes1 is a basic helix-loop-helix (bHLH) transcriptional repressor that inhibits differentiation in many cell types by repressing the transcription of other bHLH transcription factors (Kageyama et al., 2000, Mol. Cells, 10:1-7). Hes1, inhibiting cell differentiation, represses the transcription of the bHLH transcriptional activator Math1. When the Notch signal is interrupted, Hes1 is not transcribed and Math1 transcription is up-regulated (van den Brink et al., 2001, Science, 294:2115-2116).

Notch signal interruption has been measured using western blots to assay the reduction of accumulation of the NICD (Kopan et al., 1996, Proc. Natl. Acad. Sci. USA, 93:1683-1688; Lewis, 2003, Biochemistry, 42:7580-7586).

Math 1 expression is associated with cell differentiation in numerous tissue types (Birminham et al., 1999, Science, 284:1837-1841; van Den Brink et al., 2001, supra; Yang et al., 2001, supra). WO 00/73764 describes the use of Math 1 for treatment of deafness, partial hearing loss, vestibular defects due to damage or loss of inner ear hair cells, osteoarthritis, and abnormal cell proliferation. WO 02/40716 describes the use of Math1 in a marker system for the diagnosis of neoplastic disease. Math1 has also been described as a marker for brain cancer (Lee et al., 2003, Cancer Res., 63:5428-5437). A recent report shows that the serine protease adipsin (ADN) maybe used as a potential biomarker for intestinal goblet cell metaplasia (Searfoss et al., 2003, J. Biol. Chem., 278:46107-46116).

There is a need for new assays of Notch pathway modulation and new assays of Notch pathway associated drug toxicity.

### SUMMARY

The present invention provides a method for identifying a compound capable of modulating the Notch pathway comprising providing an animal or a cell in culture, administering to the animal a test compound or contacting the cell with a test compound, and detecting Math1 expression in a sample from the animal or the cell, wherein a change in Math1 expression in the presence of said compound compared with Math1 expression in the absence of said compound indicates that said compound modulates the Notch pathway.

In some embodiments, detecting is achieved by measuring the amount of Math1 protein, the amount of Math1 mRNA, or the level of Math1 activity. In some embodiments, detecting is performed on a sample obtained from an animal and the sample is selected from tissue, blood, plasma, serum, stool, urine, saliva, tears, and semen. In further embodiments, detecting is achieved by measuring the amount of Math1 protein. In further embodiments, detecting is performed on a sample from the cell in culture, said sample selected from cells, cell lines, and conditioned cell culture media.

In another aspect, the present invention provides a method for detecting modulation of the Notch pathway comprising detecting an alteration in the expression of Math1. In some embodiments, detecting is achieved by comparing Math1 expression levels of different samples.

### BRIEF DESCRIPTION OF THE DRAWINGS

FIGS. 1A and 1B show photos of sections of duodenum from control (1A) and NPMC-treated (1B) rats taken at 400× magnification.

FIG. 2 is a line graph showing the relationship of Hes1 and Math1 transcription in the duodenum upon treatment with NPMC in a 5 days time course.

FIG. 3 presents four line graphs showing a comparison of Math1 and adipsin transcription as it relates to effects on Hes1 transcription in response to four different NPMCs (A, B, C, and D) of varying efficacies.

FIG. 4 shows photographs of chemiluminescent filters depicting the immunoblot detection of Math1 protein in fecal extracts of rats treated with two different NPMCs of different efficacies. Each lane contained 25μg of protein from fecal extracts.

### DETAILED DESCRIPTION

The present invention is based upon our discovery of an association between Math1 transcript and protein levels and drug treatment. We have specifically found a correlation between Math1 transcript and protein levels and treatment with Notch pathway modulating compounds (NPMCs). Specifically, we treated rats with three different gamma secretase inhibitors (known to interrupt the Notch signal), and using gene expression profiling, we showed an induction of Math1 expression in intestinal tissue. Additionally, we have found increased Math1 protein levels in the feces of rats treated with compounds that interrupt the Notch signal.

Our discovery can be harnessed in assays that use Math1 as a biomarker of Notch pathway modulation. Notch pathway modulation, particularly Notch pathway interruption, can serve as an indicator of compound toxicity. The present invention provides methods for detecting modulation of the Notch pathway comprising detecting an alteration in the expression of Math1. In general, detecting an alteration in the expression of Math1 is achieved by comparing Math1 expression levels of different samples.

Determining the efficacy or toxicity of a NPMC is made difficult by the lack of a reliable biomarker. The ability to use Math1 expression as a marker of Notch pathway modulation is the basis of novel assays for screening potential therapeutic drugs in animals and for the clinician to prevent deleterious side effects in patients. The present invention provides assays based upon the detecting or measuring of Math1 encoding polynucleotides (nucleic acid) and/or Math1 polypeptides in samples for the detection of modulation of the Notch pathway.

The assays of the present invention can be used to test the results of drug treatment or administration on whole animals, or cells in tissue culture. The assays of the present invention are useful in reducing the time and effort in the determination of which drug candidates should be removed from development (those having undesirable effects) and which drug candidates should be advanced in development (those having desirable effects). The assays of the present invention can be used to identify compounds that modulate the Notch pathway.

In one aspect, the present invention provides assays for identifying compounds that modulate the Notch pathway. The assays detect or measure the level of Math1 expression in the presence and in the absence of a test compound, and the levels in the presence and absence of the test compound are compared. An alteration in the level or amount of Math1 expression in the presence of a test compound as compared to in the absence of a test compound is indicative that the test compound is capable of modulating the Notch pathway. Increased Math1 expression level or amount in the presence of a test compound is indicative of a blocking or interruption of the Notch pathway. Decreased Math1 expression level or amount in the presence of a test compound is indicative of an activation or enhancement of the Notch pathway.

As used herein, the terms “modulate” or “modulates” in reference to the Notch pathway include any alteration, either an inhibition or enhancement, of the Notch pathway. Assays of the present invention utilize the measurement of Math1 expression levels as the basis for detecting Notch pathway modulation. Any measurable change in the level or amount of Math1 expression can be correlated to a modulation of the Notch pathway.

In some embodiments of the present invention, the measurements of Math1 expression are performed on or carried out on samples.

As used herein, the term “sample” includes any product of biological origin, including, but not limited to, cells, cell lines, cell culture media, and biological tissue. Samples include, but are not limited to, tissue, including biopsy and autopsy tissue, blood, blood products such as plasma or serum, stool (fecal material), urine, saliva, tears, and semen. Cell culture media is media that has been conditioned with cells or cell lines, i.e., media in which cells or cell lines have been cultured.

In some embodiments of the present invention, Math1 expression is measured in a sample obtained from an animal.

In some embodiments of the present invention, an animal is treated with or administered test compounds, and following such treatment or administration, samples are taken from the animal and Math1 expression is measured, and compared to Math1 expression in control samples. Control samples can be samples taken from the same animal in the absence of test compounds, or from other control animals that have not been treated with or administered test compounds. Those of skill in the art will recognize many methods of establishing or generating such control samples.

In some embodiments of the present invention, the sample is selected from tissue, blood, plasma, serum, stool, urine, saliva, tears, and semen.

In particular embodiments of the present invention, the sample is stool.

in some embodiments of the present invention, cells in culture are exposed to, treated with or administered test compounds, and following such exposure, treatment or administration, samples are taken from the cell culture and Math1 expression is measured, and compared to Math1 expression in control samples, such as untreated cells. Those of skill in the art will recognize many methods of establishing or generating such control samples.

In some embodiments of the present invention, Math1 levels are measured in cells, cell lines, or conditioned cell culture media.

As used herein, the term “expression” in reference to Math1 refers to all indicators of transcriptional expression of the Math1 encoding gene. Such indicators include Math1 transcript products, including MRNA, generated as a result of transcription of the Math1 gene, translation products, including all forms of Math1 polypeptide or protein and fragments or peptides thereof, generated as a result of translation of Math1 transcripts, and demonstrable or otherwise measurable Math1 activity. The measurement and/or quantitation of Math1 transcript or mRNA, Math1 polypeptide, protein, or fragments or peptides thereof, and Math1 activity is indicative of “Math1 expression.”

In some embodiments of the present invention, measuring of Math1 expression levels is achieved by assaying the amount of Math1 protein, the amount of Math1 mRNA, or the level of Math1 activity.

Math1 transcripts or mRNA can be measured using any of many techniques known to those of skill in the art, including, but not limited to, northern hybridization, PCR, reverse transcription followed by PCR, quantitative real-time PCR, nuclease protection assay, and in situ hybridization.

Math1 protein can be measured by many standard techniques known to those of skill in art, including, but not limited to, immunoassays using a Math1 specific antibody in an enzyme linked immunosorbent assay (ELISA) and western immunoblotting. Math1 protein levels can also be determined using a Math1 specific antibody or mass spectroscopy in conjunction with 2 dimensional gel electrophoresis (separation of proteins by their isoelectric point (IEF) in the first dimension followed by molecular weight determination using sodium dodecyl sulphate polyacrylamide gel electrophoresis (SDS-PAGE)).

Math1 activity can be measured by a variety of assays known to those of skill in the art. Any of these assays can be used to measure Math1 activity levels in the assays of the present invention. For example, transcription factor activity can be measured by gel retardation assays. Available assays determine if a putative factor binds to DNA and on what nucleotide sequence it binds. See, for example, McKay et al., 1998, Analyt. Biochem., 265:28-34.

In some embodiments of the present invention, Math1 transcript levels in a sample are measured by quantitative real-time reverse transcription PCR.

In some embodiments of the present invention, Math1 transcript levels are measured by Northern blot.

In other embodiments of the present invention, Math1 transcript levels in a sample are determined by nuclease protection assay.

In further embodiments of the present invention, Math1 protein levels are determined by western blot using a Math1 specific antibody.

In still further embodiments of the present invention, Math1 protein levels are determined by radioimmunoassay (RIA).

In some embodiments of the present invention, Math1 protein levels are determined by radioligand binding.

In other embodiments of the present invention, Math1 protein levels are determined by liquid chromatography.

Any compounds can be tested using the methods of the present invention. Potential test compounds include, but are not limited to, biologically active compounds in classes of compounds suspected of having or known to have side effects or modes of action that interrupt or potentiate the Notch pathway. These agents include, but are not limited to, small molecules (Beher & Shearman, 2002, Biochem. Soc. Trans., 30:534-537; Wolfe et al., 1998, J. Med. Chem., 41:6-9; Netzer et al., 2003, Proc. Natl. Acad. Sci. USA, 100:12444-12449), antibodies directed against Notch or a member of the Notch pathway, and nucleic acids encoding proteins that may constituitively activate or interrupt the Notch pathway by expression of a wild-type or mutant form of a protein that is known to modulate the Notch pathway.(Zlobin et al., 2000,Cur. Pharma. Biotech., 1:83-106).

Members of the Notch pathway include, but are not limited to, Notch ligands such as Delta, Serrate and Lag2 and their mammalian homologs, enzymes that are known to process these ligands such as elements from the Fringe family and the metalloproteinase Kuzbanian, elements known to be involved in Notch processing such as furin, TACE/ADAM10 and the γ-secretase complex, downstream effector molecules such as Su(H)/CBF1, and members of the Hes family of bHLH transcription factors.

The invention is further illustrated by way of the following examples, which are intended to elaborate several embodiments of the invention. These examples are not intended to, nor are they to be construed to, limit the scope of the invention. It will be clear that the invention may be practiced otherwise than as particularly described herein. Numerous modifications and variations of the present invention are possible in view of the teachings herein and, therefore, are within the scope of the invention.

### EXAMPLES

### Example 1

### Detection of Math1 Transcript by Quantitative Real-time PCR.

### Summary

We dosed Han Wistar rats with 3 gamma secretase (γ-sec) inhibitors intraperitoneally at 0, 10, 30 and 100 μmol/kg/bid for 5 days with a benzodiazepine (BD), and a dibenzazepine (DBZ), both known to interrupt the Notch signal, and an arylsulfonamide (AS) that shows weaker Notch signal interruption. Using gene expression profiling we detected several deregulated factors that are consistent with Hes-1 KO data. We confirmed the up-regulation of ADN mRNA in intestinal tissue by DBZ and BZ treatment but not AS treatment. However, the induction of Math1 was markedly higher than ADN at earlier time points and at lower dose with DBZ and BZ. This up-regulation preceded the appearance of goblet cell metaplasia in the crypts. Also, these elements showed a temporally distinct response to γ-sec inhibition.

### Method

Compounds with varying potency for modulating the Notch1 pathway and causing intestinal metaplasia were injected intraperitoneally into rats twice daily for 5 days at a concentration of 10 μmol/kg, 30 μmol/kg and 100 μmol/kg. A section of duodenum was taken from untreated rats and those treated with compound. Samples were immediately frozen in liquid nitrogen then placed in a freezer maintained at −80° C. for preservation. RNA was extracted form these samples using the RNeasy Midi Kit obtained from Qiagen (Valencia, Calif.) according to the manufacturer's protocol for isolation of RNA from animal tissue. Five micrograms total RNA was reverse transcribed in a 50 μl reaction using random hexamer oligonucleotides and Superscript II reverse transcription system from Invitrogen Inc. (Carlsbad, Calif.). Assuming complete conversion of RNA to cDNA the reverse transcription reactions were diluted to 25 ng/μl.

Quantitative real-time PCR (QRT-PCR) was performed by creating a standard curve assaying the gene of interest at known cDNA quantities of 50 ng, 25 ng, 8.33 ng, 2.76 ng, 0.910 ng and 0.154 ng. Each sample was then assayed for mRNA abundance of Math1 and Hes1 using 1 μl at 25 ng/μl cDNA (Table 1). Biosource International (Camarillo, Calif.) synthesized oligonucleotide primers and fluorescence resonance energy transfer (FRET) probes. The 50 μl Math1 QRT-PCRs contained 25 ng template cDNA, 200 nM each primer, 100 nM FRET probe and 25 μl Taqman Universal Master Mix obtained from Applied Biosystems (Foster City, Calif.). The 50 μl Hes1 reactions contained 25 ng template. 400 nM each primer, 100 nM FRET probe and 25 μl Taqman Universal Master Mix. Thermo cycling was performed on a DNA Engine Opticon 2 manufactured by MJ Research (Waltham Mass.) using the following profile: 50° C. 2min, 94° C. 10 min., then 40 cycles of 94° C. 15 sec., 60° C. 1 min. The raw data were applied to the standard curve and quantities were extrapolated using Prism by GraphPad Software (San Diego, Calif.). These quantities were then transformed to express fold change relative to the vehicle control (VC).

### Results

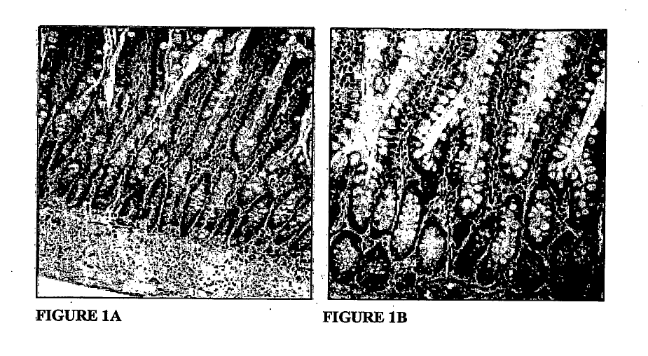

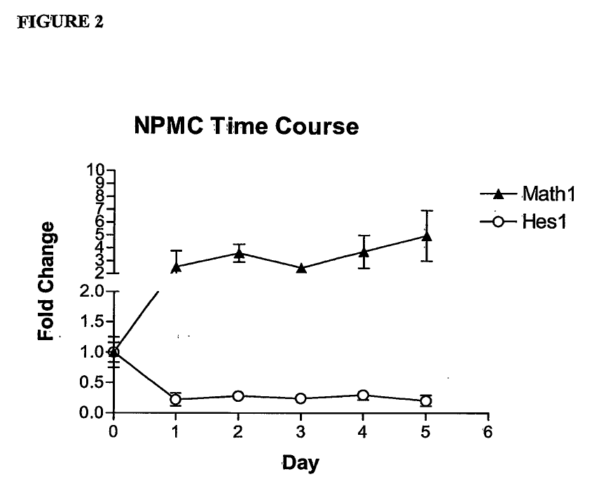

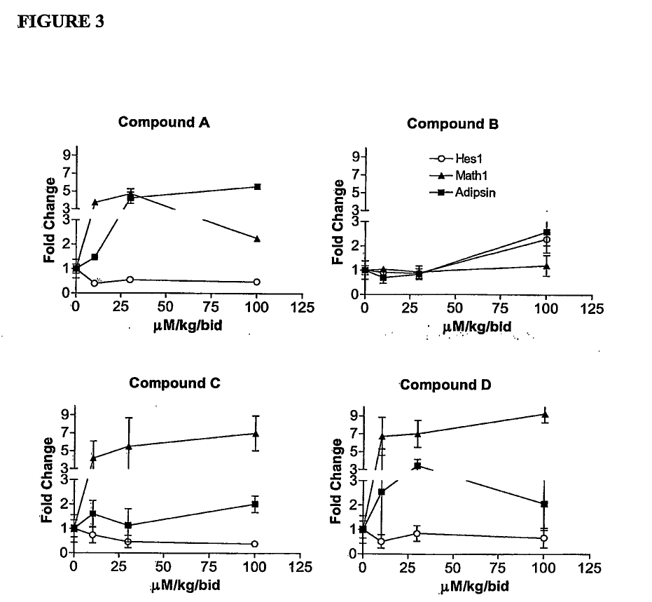

Upon twice-daily, interperitoneal injection with a NPMC in Han Wistar rats, the primary pathology found was an intestinal goblet cell metaplasia (FIG. 1B). These findings were analogous to Hes1 knockout data that show a metaplastic condition in the embryonic gut (Jensen et al. 2003, supra). Seeing a similar phenomenon, we investigated the relationship between Math1 and Hes1 in animals with intestinal goblet cell metaplasia resulting from treatment with NPMCs. The data clearly show that 24 hours after administration, Math1 transcript abundance increases and Hes1 transcript decreases (FIG. 2). Intestinal goblet cell metaplasia is not seen in the duodenum until day 2. Therefore, the up-regulation of Math1 transcript is predictive of intestinal goblet cell metaplasia caused by treatment with NPMCs. We also clearly demonstrate that affecting Hes1 transcription by interruption of the Notch pathway is not always followed by a predictable change in adipsin transcription (FIG. 3). However, when considering the compounds tested, Math1 transcription is up-regulated upon Hes1 down-regulation and unchanged when Hes1 is unchanged or up-regulated.

TABLE 1

Oligos used to detect adipsin, Hes1 (Searfoss et
al., 2003 supra) and Math1 transcripts

Adipsin

| | | |
|---|---|---|
| Forward | 5′-GGGCAATCACCAAGAACATGAT-3′ | SEQ ID NO: 1 |
| Reverse | 5′-GGAGTCGCCCCTGCAAGT-3′ | SEQ ID NO: 2 |
| Probe | 5′FAM-TGTGCAGAGAGCAACCGCAGGG-TAMRA3′ | SEQ ID NO: 3 |

Hes1

| | | |
|---|---|---|
| Forward | 5′-TACCCCAGCCAGTGTCAACA-3′ | SEQ ID NO: 4 |
| Reverse | 5′-TCCATGATAGGCTTTGATGACTTTC-3′ | SEQ ID NO: 5 |
| Probe | 5′FAM-CGGGACAAACCAAAGACAGCCTCTGA-TAMRA3′ | SEQ ID NO: 6 |

Math 1

| | | |
|---|---|---|
| Forward | 5′-AGCTGGACGCTTTGCACTTT-3′ | SEQ ID NO: 7 |
| Reverse | 5′-TCTGTGCCATCATCGCTGTT-3′ | SEQ ID NO: 8 |
| Probe | 5′FAM-CAGCTTTCGAGGACCGGGCCC-TAMRA3′ | SEQ ID NO: 9 |

### Example 2

### Detection of Math1 Protein in Fecal Extracts by Western Blot.

### Methods

Fecal material was collected from cages housing rats that were treated with NPMC or vehicle alone. Individual stool samples were placed in 12 mm round bottom tubes containing freshly prepared 1-2 mL TBS, 0.1% Tween 20. Protease inhibitor sets II and III were added to 2× final concentration from Calbiochem (La Jolla, Calif.). The feces samples were mixed by pipetting to roughly disperse them into solution. This suspension was homogenized on ice using a Power Gen 1800 G homogenizer with a 7 mm×95 mm probe for 30 seconds (Fisher Pittsburg, Pa.). These samples were centrifuged at 500× g for 10-15 minutes at 4° C. in a Sorvall RT centrifuge. The supernatant was collected and the protein content was quantitated.

Protein samples were mixed with LDS sample loading dye and reducing agent (Invitrogen Carlsbad, Calif.). An adjusted volume of this mixture containing 12.5 mg of protein was loaded onto 4-12% gradient MES acrylamide gel. The gel was run at 200 volts for 1 hour. The protein was transferred to a PVDF membrane. After transfer the membrane was placed in Tris buffered saline pH 7.4 containing 0.1% Tween 20 and 1% bovine serum albumin (TBST-BSA) and stored at 4° C. overnight. The following day the PVDF membrane was placed in TBST-BSA containing the primary antibody, a 1:1000 dilution of a rabbit. anti-human Math1 antibody (catalog A3950 Lot L3052158 US Biologicals Swampscott, Mass.) and incubated for 1 hour at room temperature with gentle agitation. The solution was removed and the membrane was washed 5 times for 5 minutes each in TBST. The membrane was incubated in TBST-BSA containing the secondary. antibody, a 1:5000 dilution of goat anti-rabbit IgG conjugated to horse radish peroxidase (HRP) (catalog ab6721 Abcam Cambridge, Mass.). The membrane was subsequently washed 5 times for 5 minutes each in TBST. A positive antibody reaction was visualized using ChemiGlow™ (Alpha Inotech San Leandro, Calif.) enhanced chemiluminescence and Alpha Innotech Imager.

### Results

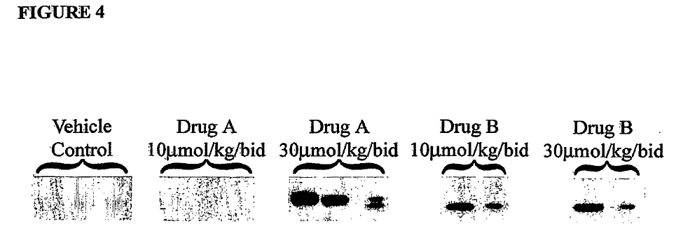

We have shown that Math1 is up-regulated upon treatment with several Notch pathway modulating compounds (NPMCs) with weak to potent efficacies (FIG. 4). The data show detection of Math1 protein in feces by day 3 following the onset of intestinal metaplasia by day 2 (Table 2).

TABLE 2
Detection of Math1 Protein in Animals in a 5 Day Time Course

| | Control | Day 1 | Day 2 | Day 3 | Day 4 | Day 5 |
|---|---|---|---|---|---|---|
| Math1 | + | + | + | ++ | +++ | +++ |

### Example 3

### Detection of Math1 Protein by ELISA.

Math1 is detected in protein from animals, cells or cell lines treated with NPMC by using an enzyme linked immunosorbent assay (ELISA).

### Example 4

### Detection of Math1 Protein by Immunoprecipitation.

Math1 is detected in protein from animals, cells or cell lines treated with NPMC by using immunoprecipitation.

### Example 5

### Detection of Math1 Protein 2D Gel and Mass Spectroscopy.

Math1 is detected in protein from animals, cells or cell lines treated with NPMC by using 2 dimensional gel electrophoresis and antibodies against Math1 or mass spectroscopy.

The foregoing examples are meant to illustrate the invention and are not to be construed to limit the invention in any way. Those skilled in the art will recognize modifications that are within the spirit and scope of the invention.

We claim:

1. A method for identifying a compound capable of modulating the Notch pathway comprising:
a) providing an animal or a cell in culture;
b) administering to the animal a test compound or contacting the cell with a test compound; and
c) detecting Math1 expression in a sample from the animal or the cell, wherein a change in Math1 expression in the presence of said compound compared with Math1 expression in the absence of said compound indicates that said compound modulates the Notch pathway.

2. The method according to claim 1, wherein said detecting is achieved by measuring the amount of Math1 protein, the amount of Math1 mRNA, or the level of Math1 activity.

3. The method according to claim 1, wherein said detecting is performed on a sample obtained from an animal and the sample is selected from tissue, blood, plasma, serum, stool, urine, saliva, tears, and semen.

4. The method according to claim 3, wherein the sample is stool.

5. The method according to claim 1, wherein said detecting is achieved by measuring the amount of Math1 protein.

6. The method according to claim 1, wherein said detecting is performed on a sample from the cell in culture, said sample selected from cells, cell lines, and conditioned cell culture media.

7. A method for detecting modulation of the Notch pathway comprising detecting an alteration in the expression of Math1.

8. The method of claim 7, wherein detecting an alteration in the expression of Math1 is achieved by comparing Math1 expression levels of different samples.


SEQUENCE LISTING (9 sequences)

SEQ ID NO: 1 - DNA, 22 bp, Artificial
  Primer
gggcaatcaccaagaacatgat

SEQ ID NO: 2 - DNA, 18 bp, Artificial
  Primer
ggagtcgcccctgcaagt

SEQ ID NO: 3 - DNA, 22 bp, Artificial
  Probe
tgtgcagagagcaaccgcaggg

SEQ ID NO: 4 - DNA, 20 bp, Artificial
  Primer
taccccagccagtgtcaaca

SEQ ID NO: 5 - DNA, 25 bp, Artificial
  Primer
tccatgataggctttgatgactttc

SEQ ID NO: 6 - DNA, 26 bp, Artificial
  Probe
ccggacaaaccaaagacagcctctga

SEQ ID NO: 7 - DNA, 20 bp, Artificial
  Primer
agctggacgctttgcacttt

SEQ ID NO: 8 - DNA, 20 bp, Artificial
  Primer
tctgtgccatcatcgctgtt

SEQ ID NO: 9 - DNA, 21 bp, Artificial
  Probe
cagctttcgaggaccgggccc